(19) United States
(12) Patent Application Publication
(10) Pub. No.: US 2006/0069263 A1
(43) Pub. Date: Mar. 30, 2006

(54) Process for the reduction of (S)-2-amino-6-propionamido-4,5,6,7-tetrahydrobenzo-thiazole

(76) Inventors: Irina Gribun, Bat Yam (IL); Ada Salman, Ramat Gan (IL); Meital Shiffer, Tel Aviv (IL); Moty Shookrun, Petach-Tikva (IL); Michael Brand, RaAnana (IL); Oded Arad, Rehovot (IL); Joseph Kaspi, Givatayim (IL)

Correspondence Address: Martin Moynihan;PRTSI, Inc., P.O. Box 16446, Arlington, VA, 22215, US

(21) Appl. No.: 11/233,096

(22) Filed: Sep. 23, 2005

Related U.S. Application Data
(60) Provisional application No. 60/614,422, filed on Sep. 30, 2004.

(51) Int. Cl. C07D 277/82

(52) U.S. Cl. 548/161

(57) ABSTRACT

A process is disclosed for the reduction of (S)-2-amino-6-propionamido-4,5,6,7-tetrahydrobenzothiazole, which comprises reacting (S)-2-amino-6-propionamido-4,5,6,7-tetrahydrobenzothiazole with a borane reagent in the presence of suitable organic solvent to yield (S)-2-amino-6-propylamino-4,5,6,7-tetrahydrobenzothiazole base, which may be converted to an acid addition salt thereof. The process provided herein can be easily, conveniently and inexpensively scaled-up.

### RELATED APPLICATIONS

The present application claims priority from U.S. Provisional Patent Application No. 60/614,422, filed on Sep. 29, 2004, which is incorporated herein by reference.

### FIELD OF THE INVENTION

The present invention relates to an improved process for the reduction of (S)-2-amino-6-propionamido-4,5,6,7-tetrahydrobenzothiazole, an intermediate useful in the preparation of (S)-2-amino-6-propylamino-4,5,6,7-tetrahydrobenzothiazole, more commonly known as pramipexole.

### BACKGROUND OF THE INVENTION

(S)-2-amino-6-propylamino-4,5,6,7-tetrahydrobenzothiazole dihydrochloride, more commonly known as pramipexole dihydrochloride, is a synthetic aminobenzothiazole derivative having the molecular formula 1, which is marketed under the trade name Mirapex®.

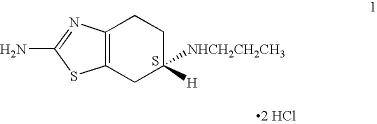

The drug is a dopamine agonist used for treating Parkinson's disease by stimulating the dopamine receptors in the brain.

Various synthetic routes for preparing pramipexole, its salts thereof and the intermediates thereof were previously described in European Patent Nos. 186087 and 207696; U.S. Pat. Nos. 6,727,367 and 6,770,761; and PCT Publications WO 2004/026850, WO 2004/041797 and WO 2005/014562. An additional synthetic route was disclosed by C. S Schneider and J. Mierau in J. Med. Chem., 1987, 30, 494-498. According to this route, pramipexole may be prepared by reacting (S)-2-amino-6-propionamido-4,5,6,7-tetrahydrobenzothiazole, a compound of formula 2, with borane tetrahydrofuran complex (BTHF) in the presence of anhydrous THF to yield (S)-2-amino-6-propylamino-4,5,6,7-tetrahydrobenzothiazole base, a compound of formula 3. The isolated base is consequently converted into the dihydrochloride salt, which is recrystallized from methanol. This process is illustrated by the following reaction scheme:

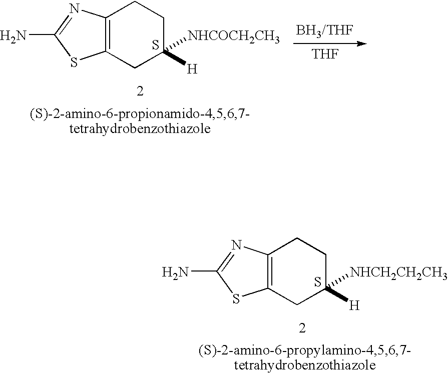

This synthetic route involves using the reducing agent BTHF, which is supplied as a 1.0 M or 1.5 M solution in THF. The reagent is thermally unstable and must be stored in the cold (below 5° C.). Furthermore, BTHF is susceptible to hydrolysis, readily reacting with water to form hydrogen and boric acid and readily reacting with atmospheric moisture upon exposure to air, resulting in a decrease in assay. At elevated temperatures of above 50° C. and in the absence of a substrate BTHF decomposes by cleavage of the ether ring to evolve the diborane gas, which is extremely toxic. In addition, tetrahydrofuran can form potentially explosive peroxides upon long standing in the air.

All the above restrictions and warnings make the use of BTHF complicated, expensive (due to high freight and storage costs), inconvenient and environmentally harmful and it appears clear that this process cannot be advantageously used for large-scale production.

The object of the present invention is to provide an improved process for the reduction of (S)-2-amino-6-propionamido-4,5,6,7-tetrahydrobenzothiazole, a compound of formula 2, which avoids using the borane tetrahydrofuran complex via an efficient, convenient and economic process by using alternative borane reagents which are more convenient to handle and more stable for synthetic applications as the reductive agent.

### SUMMARY OF THE INVENTION

In one aspect, the present invention provides an improved process for the reduction of (S)-2-amino-6-propionamido-4,5,6,7-tetrahydrobenzothiazole, a compound of formula 2, which comprises reacting the said compound of formula 2 with a borane reagent in the presence of a suitable organic solvent to yield (S)-2-amino-6-propylamino-4,5,6,7-tetrahydrobenzothiazole base.

In another aspect of the present invention, once the reaction is complete, the product may be isolated as a free base or as an acid addition salt.

In another aspect of the present invention, once the reaction is complete the product as a free base, that is compound of formula 3, can be conveniently separated from impurities such as unreacted starting material, organic and inorganic salts and side-products by conventional physical separation (such as filtration, extraction, etc) of the impurities from the reaction mixture.

In yet another aspect of the present invention, once the reaction is complete, the said product may be converted into an acid addition salt and isolated in solid state by methods described hereinabove.

In yet another aspect of the present invention there is provided a process for preparing pramipexole dihydrochloride, compound of formula 1, by converting the said product to pramipexole dihydrochloride using any of the conventional methods known in the art.

### DESCRIPTION OF THE PREFERRED EMBODIMENTS

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The present invention provides an improved process for the reduction of (S)-2-amino-6-propionamido-4,5,6,7-tetrahydrobenzothiazole, a compound of formula 2, which avoids using borane tetrahydrofuran complex.

The compound of formula 2, used as the starting material in the embodiments disclosed hereinafter is known and obtainable e.g. by conventional methods known in the art.

In accordance with the present invention, an improved process is provided for the reduction of (S)-2-amino-6-propionamido-4,5,6,7-tetrahydrobenzothiazole that avoids using borane tetrahydrofuran complex, comprising the steps of:
a. reacting (S)-2-amino-6-propionamido-4,5,6,7-tetrahydrobenzothiazole, with a convenient borane reagent in the presence of a suitable organic solvent to thereby obtain (S)-2-amino-6-propylamino-4,5,6,7-tetrahydrobenzothiazole base;
b. isolating the (S)-2-amino-6-propylamino-4,5,6,7-tetrahydrobenzothiazole as a free base or as an acid addition salt thereof,
c. optionally purifying the pramipexole acid addition salt.

In one embodiment of the present invention, (S)-2-amino-6-propionamido-4,5,6,7-tetrahydrobenzothiazole is reacted with a borane reagent. Usable borane reagents in the context of the present invention include, but are not limited to, borane dimethyl sulfide complex (BDMS), borane-N-ethyl-N-isopropylaniline complex, borane isoamyl sulfide complex, monochloroborane dioxane complex and dichloroborane dioxane complex.

In a preferred embodiment of the present invention, (S)-2-amino-6-propionamido-4,5,6,7-tetrahydrobenzothiazole is reacted with borane dimethyl sulfide complex.

In another embodiment of the present invention, (S)-2-amino-6-propionamido-4,5,6,7-tetrahydrobenzothiazole is reacted with a borane reagent in the presence of a suitable organic solvent. Preferably, the organic solvent is inert, water free ether, which does not contain a cationically polymerizable carbon-carbon unsaturated bond. Thus, according to the present invention, the ether is a compound containing 2 to 10 carbon atoms and one or two oxygen atoms, wherein the ring structure forming the cyclic ether has no unsaturated bond and the ether ring may further have a substituent such as an alkyl group, an alkoxy group or an aryl group.

In another embodiment of the present invention, the solvent may be selected from the group consisting of cyclic ethers such as tetrahydrofuran, 2-methyltetrahydrofurane, trimethylene oxide (oxetane), pentamethylene oxide (oxane), 1,4-dioxane etc. and chain ethers such as diethyl ether, ethyl propyl ether, isopropyl methyl ether, dipropyl ether, t-butyl propyl ether and the like and mixtures thereof.

In another preferred embodiment of the present invention, (S)-2-amino-6-propionamido-4,5,6,7-tetrahydrobenzothiazole is reacted with borane reagent in the presence of tetrahydrofuran, 2-methyltetrahydrofurane or diethyl ether or mixtures thereof.

In another embodiment of the present invention, the reaction is conveniently conducted at ambient temperature or at an elevated temperature, preferably at a temperature between ambient and the reflux temperature of the solvent.

In another embodiment of the present invention, the reaction is carried out for an extended period of time, preferably from about 1 hour to about several days, more preferably from about 1 hour to about 5 hours.

In yet another embodiment of the present invention, when complete, the reaction is quenched by the careful addition of a suitable quenching solvent such as alcohol and aqueous acid solution, such as 1 M hydrochloric acid solution.

In another embodiment of the present invention, (S)-2-amino-6-propylamino-4,5,6,7-tetrahydrobenzothiazole base may be converted to an acid addition salt without isolation of the free base, i.e. in the same reaction vessel. Preferably, these salts are pharmaceutically acceptable salts.

In yet another embodiment of the present invention, the conversion is accomplished by treatment with at least a stoichiometric amount of an appropriate acid. In the present invention, the appropriate acid includes, but is not limited to inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. The preferred salts are the tartrate and hydrochloride salts.

In yet another embodiment of the present invention, the product may be isolated as a free base or as an acid addition salt by conventional techniques well-known in the art, while filtration and extraction or a combination of these procedures are the most preferred methods.

In yet another embodiment of the present invention, to assist in impurity removal, it is effective to treat the substrate with an adsorbent, preferably with active charcoal. Activated charcoal is added to the (S)-2-amino-6-propylamino-4,5,6,7-tetrahydrobenzothiazole base mixture. If desired, a filter-aid may be additionally added. After the activated charcoal has been added, stirring is continued at constant temperature for between 5 and 60 minutes, preferably between 10 and 30 minutes, most preferably about 15 minutes, and the mixture obtained is filtered to remove the solids.

In yet another embodiment of the present invention, the isolated product can be dried using conventionally known methods to give pure pramipexole as a free base or as an acid addition salt. The drying stage may be carried out by increasing the temperature or reducing the pressure or a combination of both. Non limiting examples of drying technologies or equipments usable in context of the present invention include vacuum ovens, tray ovens, rotary ovens and fluidized bed dryers.

In another embodiment of the present invention, the isolated product, either as a free base or as the acid addition salt thereof, may be further treated with an odor reducing agent.

Odor reducing agents usable in the context of the present invention include, but are not limited to, hydrogen peroxide, halogen donors such as chlorine dioxide, sodium hypochlorite, sodium periodate, sodium perchlorite and hypobromous acids and the like. A preferred odor reducing agent is hydrogen peroxide.

Thus, the present invention uses an odor reducing agent, preferably an aqueous hydrogen peroxide, which completely eliminates the odor when contacted with the reaction product containing the odor and/or noxious components.

In another embodiment of the present invention, the isolated product as a free base or as the acid addition salt thereof may be converted to pramipexole dihydrochloride by any convenient method known in the art.

In another embodiment of the present invention, if the produced pramipexole dihydrochloride is not sufficiently pure, it may be further purified by recrystallization or by converting the acid addition salts to the corresponding free base by treatment with at least a stoichiometric equivalent of a suitable organic or inorganic base such as described hereinabove and converting the pure free base product again to a corresponding acid addition salt.

In another embodiment of the present invention, the crystalline pramipexole dihydrochloride may be recrystallized by any conventional recrystallization method known in the art.

In yet another embodiment of the present invention, the processes described hereinabove for the reduction of (S)-2-amino-6-propionamido-4,5,6,7-tetrahydrobenzothiazole may be conveniently and inexpensively scaled-up.

Although, the following examples illustrate the practice of the present invention in some of its embodiments, the examples should not be construed as limiting the scope of the invention. Other embodiments will be apparent to one skilled in the art from consideration of the specification and examples. It is intended that the specification, including the examples, is considered exemplary only, with the scope and spirit of the invention being indicated by the claims which follow.

### EXAMPLES

### Example 1

A 250 ml reaction vessel equipped with a magnetic stirrer, nitrogen inlet and a reflux condenser was charged with 50 ml of dry 2-methyltetrahydrofuran and 15.5 ml of borane dimethyl sulfide complex and the solution was stirred at room temperature. 4.6 g of (S)-2-amino-6-propionamido-4,5,6,7-tetrahydrobenzothiazole were added in portions. The reaction mixture was heated to 50° C. to afford a clear solution. After 2 hours the reaction mixture was cooled to 5° C. and 6.7 ml of methanol were added in portions while maintaining the temperature at 5° C. A mixture of 11 ml of water and 15.7 ml of HCl solution (32%) were added to afford a suspension. The reaction mixture was allowed to warm to room temperature and 30.9 ml of aqueous sodium hydroxide (25%) solution were added in portions followed by addition of 42 ml of 2-methyltetrahydrofuran. The reaction mixture was heated to 50° C. to afford a two phase system. The reaction mixture was cooled and the layers were separated. The organic phase was washed with 2×20 ml of water. Activated charcoal was added to the organic layer containing the product as a free base. The suspension thus obtained was stirred for at least 15 minutes at elevated temperature (between 40° C. and 50° C.) and then hot-filtered. The filter was rinsed with 20 ml of warm 2-methyltetrahydrofurane.

The organic phase was heated to reflux and about 80 ml of 2-methyltetrahydrofurane were distilled out at atmospheric pressure. 20 ml of ethanol and about 13 ml of 14.6% solution of HCl in isopropanol were added and the reaction mixture was stirred for 1 hour at room temperature. The mixture was cooled to 5° C. and stirred for additional 1 hour. The precipitate was filtered, washed with cold ethanol and dried at 60° C. to yield 4.36 g (77%) of the desired product.

### Example 2

A 250 ml reaction vessel equipped with a magnetic stirrer, nitrogen inlet and a reflux condenser was charged with 50 ml of dry tetrahydrofuran and 15.5 ml of borane dimethyl sulfide complex and the solution was stirred at room temperature. 4.6 g of (S)-2-amino-6-propionamido-4,5,6,7-tetrahydrobenzothiazole were added in portions. The reaction mixture was heated to 50° C. to afford a clear solution. After 2 hours the reaction mixture was cooled to 5° C. and 6.7 ml of methanol were added in portions while maintaining the temperature at 5° C. A mixture of 11 ml of water and 15.7 ml of HCl solution (32%) were added to afford a suspension. The reaction mixture was heated to reflux and the majority of the tetrahydrofuran-water mixture was distilled out at atmospheric pressure to a final volume of about 20 ml. The reaction mixture was allowed to cool to room temperature and about 31 ml of aqueous sodium hydroxide (25%) solution were added in portions followed by addition of 92 ml of ethyl acetate. The reaction mixture was stirred at room temperature for 15 minutes to afford a two phase system. The layers were separated and the organic phase was washed with 2×20 ml of water. Activated charcoal was added to the organic layer containing the product as a free base. The suspension thus obtained was stirred for at least 15 minutes at elevated temperature and then hot-filtered. The filter was rinsed with 20 ml of warm ethyl acetate. The reaction mixture was heated to reflux and most of the ethyl acetate was distilled out. The reaction mixture was allowed to cool to room temperature and 20 ml of absolute ethanol and about 13 ml of 14.6% solution of HCl in isopropanol were added and the reaction mixture was stirred for 1 hour at room temperature. The mixture was cooled to 5° C. and stirred for additional 1 hour. The precipitate was filtered, washed with cold 1:1 mixture of ethanol-ethyl acetate and dried at 60° C. to yield 4 g (70%) of the desired product.

### Example 3

A 250 ml reaction vessel equipped with a magnetic stirrer, nitrogen inlet and a reflux condenser was charged with 50 ml of dry tetrahydrofuran and about 14 ml of borane dimethyl sulfide complex and the solution was stirred at room temperature. 4.6 g of (S)-2-amino-6-propionamido-4,5,6,7-tetrahydrobenzothiazole were added in portions. The reaction mixture was heated to 50° C. to afford a clear solution. After 1 hour the reaction mixture was cooled to room temperature and a mixture of 10 ml of water and 20 ml of HCl solution (32%) were added to afford a suspension. The reaction mixture was heated to about 60° C. and the majority of the THF-water mixture was distilled out under vacuum. 60 ml of aqueous sodium hydroxide (25%) solution were added in portions and the reaction mixture was stirred at room temperature for 1 hour. The mixture was cooled to 5° C. and stirred for additional 1 hour. The precipitate was filtered, washed with cold water and dried at 60° C. under vacuum to yield 3.0 g (71%) of the desired product.

### Example 4

A 100 ml reaction vessel equipped with a magnetic stirrer was charged with 5.0 g of S-Pramipexole dihydrochloride and 37 ml of water. 2 ml of 30% aqueous hydrogen peroxide solution were added and the reaction mixture was stirred at room temperature. After 1 hour 13 ml of 45% potassium hydroxide solution were added in portions and the suspension was stirred at room temperature for 1 hour. The precipitate was filtered, washed with cold water and dried at 60° C. under vacuum to yield 3.0 g (86%) of S-Pramipexole base.

### Example 5

A 50 ml reaction vessel equipped with a magnetic stirrer was charged with 2.15 g of S-Pramipexole dihydrochloride and 16 ml of water. 5.6 ml of 45% potassium hydroxide solution were added in portions and the suspension was stirred at room temperature for 1 hour. The precipitate was filtered, washed with cold water and dried at 60° C. under vacuum to yield 1.3 g (86%) of Pramipexole base.

### Example 6

A 50 ml reaction vessel equipped with a magnetic stirrer was charged with 2.53 g of S-Pramipexole base and 20 ml of absolute ethanol. The reaction mixture was stirred at room temperature to afford a clear solution. The reaction mixture was filtered and the filtrate was transferred to a 50 ml reaction vessel. 7.8 ml of 14.6% solution of HCl in isopropanol were added in portions and the resulting reaction mixture was stirred for 1 hour. The mixture was cooled to 5° C. and stirred for additional 1 hour. The precipitate was filtered, washed with cold ethanol and dried at 60° C. under vacuum to yield 3.0 g (89%) of the desired product.

What is claimed is:

1. An improved process for the reduction of (S)-2-amino-6-propionamido-4,5,6,7-tetrahydrobenzothiazole which avoids using borane tetrahydrofuran complex, the process comprising:
a. reacting (S)-2-amino-6-propionamido-4,5,6,7-tetrahydrobenzothiazole, a compound of formula 2, with a convenient borane reagent in the presence of suitable organic solvent to thereby obtain (S)-2-amino-6-propylamino-4,5,6,7-tetrahydrobenzo-thiazole base;
b. isolating the (S)-2-amino-6-propylamino-4,5,6,7-tetrahydrobenzothiazole as a free base or as an acid addition salt thereof; and
c. optionally purifying the pramipexole acid addition salt.

2. The process according to claim 1, wherein said borane reagent is selected from the group consisting of borane dimethyl sulfide complex (BDMS), borane-N-ethyl-N-isopropylaniline complex, borane isoamyl sulfide complex, monochloroborane dioxane complex and dichloroborane dioxane complex.

3. The process according to claim 2, wherein the borane reagent is borane dimethyl sulfide complex.

4. The process according to claim 1, wherein the suitable organic solvent is selected from the group consisting of cyclic ethers such as tetrahydrofuran, 2-methyltetrahydrofurane, trimethylene oxide (oxetane), pentamethylene oxide (oxane), 1,4-dioxane etc. and chain ethers such as diethyl ether, ethyl propyl ether, isopropyl methyl ether, dipropyl ether, t-butyl propyl ether and the like and mixtures thereof.

5. The process according to claim 4, wherein the suitable organic solvent is tetrahydrofuran, 2-methyltetrahydrofurane or diethyl ether and mixtures thereof.

6. The process according to claim 5, wherein 2-methyltetrahydrofuran is water free.

7. The process according to claim 1, wherein said step (a) is conducted at an ambient temperature or at an elevated temperature, preferably at a temperature between ambient and the reflux temperature of the solvent.

8. The process according to claim 1, wherein said step (a) is carried out for an extended period of time, preferably from about 1 hour to about several days, more preferably from about 1 hour to about 5 hours.

9. The process according to claim 1, wherein said reaction is quenched by adding a suitable quenching solvent such as alcohol and aqueous acid solution, such as 1 M hydrochloric acid solution.

10. The process according to claim 1, wherein obtaining the acid addition salt, preferably a pharmaceutically acceptable salt, comprises reacting the (S)-2-amino-6-propylamino-4,5,6,7-tetrahydro-benzothiazole base with an appropriate acid, thus isolating the acid addition salt thereof.

11. The process according to claim 10, wherein said (S)-2-amino-6-propylamino-4,5,6,7-tetrahydrobenzothiazole base is treated with at least a stoichiometric amount of the appropriate acid.

12. The process according to claim 11, wherein said appropriate acid is selected from the group consisting of inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

13. The process according to claim 12, wherein the salts are the tartrate salt and the hydrochloride salt.

14. The process according to claim 1, wherein said isolated product, as a free base or as the acid addition salt thereof, is further treated with an odor reducing agent.

15. The process according to claim 14, wherein the odor reducing agent is selected from the group consisting of hydrogen peroxide, halogen donors such as chlorine dioxide, sodium hypochlorite, sodium periodate, sodium perchlorite and hypobromous acids and the like.

16. The process according to claim 15, wherein said odor reducing agent is hydrogen peroxide.

17. The process according to claim 13, wherein pramipexole dihydrochloride is purified to obtain a pharmaceutically pure product, the process comprising:
a. suspending pramipexole dihydrochloride in an organic solvent in the presence of organic or inorganic base optionally dissolved in water, to thereby obtain a pramipexole free base;
b. isolating said pure pramipexole free base;
c. optionally crystallizing the said base and isolating the crystallized product;
d. re-converting the said crystallized product to pramipexole dihydrochloride; and
e. optionally recrystallizing the pramipexole dihydrochloride.

18. The process according to claim 17, wherein the said process may be conveniently and inexpensively scaled-up.
